(19) United States
(12) Patent Application Publication
(10) Pub. No.: US 2018/0325397 A1
(43) Pub. Date: Nov. 15, 2018

(54) PHOTOPLETHYSMOGRAPHY DEVICE

(71) Applicant: KONINKLIJKE PHILIPS N.V., EINDHOVEN (NL)

(72) Inventors: Cristian Nicolae Presura, Veldhoven (NL); Andrei Nicolae, Eindhoven (NL); Michel Jacobi, Hasselt (BE)

(21) Appl. No.: 15/774,174

(22) PCT Filed: Nov. 2, 2016
(86) PCT No.: PCT/EP2016/076313

§ 371 Date: May 7, 2018

(30) Foreign Application Priority Data
Nov. 10, 2015 (EP) 15193879.2

(51) Int. Cl. A61B 5/0225; A61B 5/024; A61B 5/0295; A61B 5/08; A61B 5/053; A61B 5/1455

(52) U.S. Cl. CPC A61B 5/02255; A61B 5/02416; A61B 5/0295; A61B 5/681; A61B 5/0535; A61B 5/14558; A61B 2562/0233; A61B 5/0806

(57) ABSTRACT

A photoplethysmography device comprises a light source configured to direct source light towards an external object; a light sensor arranged and configured to provide a sensor signal indicative of an intensity of a first source light fraction, which has been scattered by the external object; a casing for housing the light source and the light sensor, and having a cover plate transparent for the source light and an outer face to be facing the external object; and an optical blocking arrangement in the casing between the at least one light source and the outer face of the cover plate and configured to block a second source light fraction on its propagation path extending from the light source to the outer face of the cover plate and from the outer face of the cover plate to the light sensor without leaving the casing.

### FIELD OF THE INVENTION

The invention relates to a photoplethysmography device.

### BACKGROUND OF THE INVENTION

A photoplethysmography (PPG) device employs an optical measurement technique to detect a change in a volume of an external object. In medical applications, this technique can be used to detect a change of an amount of blood in an organ or other body part of a subject. Other information that can be detected in medical applications of PPG relates to a saturation level of blood oxygen, a respiratory rate, a pulse rate, or blood pressure. In other words, PPG can be used to monitor vital-sign information of a subject.

US 2011/0130638 A1 describes a bandage-style PPG device for providing vital-sign information in pulse oximetry measurements. Light sources and light sensors are disposed on a tissue-contacting surface of a sensor body. The light sensors are provided with surface features for reducing an amount of shunted light that impinges the light sensors without having first passed through tissue. The surface features influence the path of light from the undesired light sources by directing such light away from the detecting elements of the sensor.

US 2015/0057511 A1 discloses an optical proximity sensor assembly which includes an optical proximity sensor with an infrared (IR) light emitting diode (LED) emitting light having an infrared wavelength, an IR photodetector sensitive to the infrared wavelength, an optical barrier blocking direct light rays from the LED to the IR photodetector and permitting reflected light rays to reach the at least one photodetector and an electronic integrated circuit with an amplifier for amplifying the signal detected by the photodetector, an analog to digital converter, LED drivers, noise reduction and ambient light cancellation circuitry, and a digital interface for communication with the microcontroller. The optical proximity sensor is accommodated on a wearable carrier. A single sensor may include a plurality of identical or different LED's, a plurality of photodiodes, or both. Also, several sensors may be placed on a person's skin along a vascular path to obtain data relating to blood flow and artery stiffness.

US 2014/0163342 A1 discloses a biosensor including light emitting elements and a light receiving element disposed on a principal surface of a wiring board, a light shielding portion disposed between a light emitting element sealing portion and a light receiving element sealing portion, a base medium having light transmitting properties, disposed in parallel with the wiring board with the light shielding portion therebetween, an adhesion layer having light transmitting properties that bonds the base medium with the light emitting element sealing portion, the light receiving element sealing portion and the light shielding portion, and a first electrocardiograph electrode attached to a principal surface of the base medium. Both end portions of the adhesion layer and both end portions of the base medium are disposed such that they overlap neither of the light receiving element sealing portion nor the light emitting element sealing portion when viewed from a direction normal to the principal surface of the wiring board.

### SUMMARY OF THE INVENTION

It is an object of the present invention to provide a PPG device that improves robustness and longevity of the PPG device while eliminating or reducing a perturbation of desired vital-sign information by an unwanted signal component.

According to the present invention, a photoplethysmography device, hereinafter PPG device is provided. The PPG device comprises
at least one light source configured to generate a beam of source light to be directed towards an external object;
at least one light sensor which is arranged and configured to provide a sensor signal indicative of an intensity of a first source light fraction, which has been scattered by the external object and detected by the light sensor;
a casing that houses the at least one light source and the at least one light sensor, the casing having a cover plate, which is transparent for the source light and which has an outer face to be facing the external object; and
an optical blocking arrangement, which is arranged in the casing between the at least one light source and the outer face of the cover plate and which is configured to block a second source light fraction on its propagation path extending from the light source to the outer face of the cover plate and from the outer face of the cover plate to the light sensor without leaving the casing.

In comparison with the PPG device known from US 2011/0130638 A1 the PPG device according to the present invention thus provides an improvement by allowing the light source and the light sensor to be arranged in a protective casing with a translucent cover plate without deteriorating a light sensor signal by the use of the cover plate. The casing protects the light source and the light sensor against physical damage, which may occur for instance by contact with an external object in motion, and against other unwanted effects of direct contact with the environment, such as chemical reactions. A useful component of the light sensor signal, which carries the vital-sign information, is thus provided with small perturbation, despite the use of the cover plate, by eliminating or at least reducing an intensity of an unwanted second source light fraction that does not reach the external object but—in absence of the optical blocking arrangement—would have reached the at least one light sensor without leaving the casing, in particular after reflection at the outer face of the cover plate of the casing. This elimination or at least reduction of the intensity of this second source light fraction in the detected signal increases the relative amount of the useful first source light fraction, which has been scattered by the external object and thus contains desired vital-sign information when it is detected by the at least one light sensor. This in turn allows extracting and evaluating the vital-sign information with high accuracy.

Thus, the PPG device of the present invention combines robustness and longevity with eliminated or reduced perturbation of the desired vital-sign information by the described unwanted PPG signal component.

It is noted for clarification that the external object does not form a part of the claimed PPG device. The external object is the object to be investigated by the PPG device. Non-limiting examples of external objects are elastic tubes, animals, and human beings, or parts thereof.

In the following, embodiments of the PPG device will be described.

Due to its robustness and longevity, the PPG device is particularly suited for private use by end customers. It may be provided as a stand-alone device for mobile use, using one or more batteries to be inserted into the casing for providing supply energy.

In one embodiment the casing is provided with a construction resembling that of a watch. Suitably, the casing may thus be provided with mounting elements for fastening a watch strap or the like.

In other embodiments, the casing has shape of a ring with extensions suitable for wearing on an arm or finger of a user.

In another embodiment, the casing is integrated into a sensor to be worn inside the ear of a user, or on the exterior part of the ear of a user. The sensor of this embodiment is for instance a part of an earbud, or of a headphone.

In other embodiments the PPG device is provided as a module for integration into a portable electronic computing device, such as a smart watch or smart phone that also provides other functionality for implementing applications such as measuring time, distance, velocity, or acceleration, or providing communication or computing capabilities. In such electronic application devices, the light source and detector are to be arranged on a bottom side, the outer face of the cover plate to be facing the arm or wrist of the user. Energy supply can be shared with other electronic components of such portable electronic computing devices.

Embodiments of the PPG device according comprise a PPG processing unit. The PPG processing unit is preferably configured to receive and process a light sensor signal indicative of a light amount detected by the at least one light sensor so as to provide the desired vital-signal information. The processing requirements and suitable processing methods implemented by such a PPG processing unit are known and need not be detailed in the present context.

In some embodiments, the PPG processing unit is not provided as an integral part of the PPG device, but as an external unit, which receives the light sensor signal via a communication link. The PPG processing unit of such embodiments can for instance be implemented by executable software code for execution by a processor of a computer. For monitoring applications in everyday life and in sports, a portable computer such as a smartphone is particularly suitable. A suitable communication link between the PPG device and the computer is a wired communication link, and even more so a wireless communication link, such as a Bluetooth link or a WLAN (Wi-Fi) link.

The optical blocking arrangement of the PPG device is arranged in the propagation path of the second source light fraction mentioned above. The blocking may be effected at any suitable point of the propagation path of the second source light fraction. Suitable points of the propagation path allow blocking the second source light fraction only, without reducing the amount of the first source light fraction which has been scattered by the external object and therefore carries the vital-sign information.

The optical blocking arrangement is particularly advantageous in embodiments where it is arranged and configured for avoiding a detection of source light subject to total reflection at an interface between the cover plate and an optically thinner ambient medium outside the casing (i.e., the outer face of the cover plate) by the light sensor. This is particularly useful in preferred embodiments with a cover plate that has an index of refraction n_{CP} larger than a typical refractive index n_{SE} of an ambient optical medium outside the casing, such as air (having a refractive index of approximately 1.0) or water (approximately 1.3). In such embodiments, the outer face of the cover plate forms an interface with the ambient optical medium outside the casing and would give rise to total reflection for source light at all angles of incidence of the source light on this interface that are larger than arcsin

$$( \frac{{n}_{SE}}{{n}_{CP}} ) .$$

Taking as a non-limiting example a cover plate made of glass (n_{CP}=1.5) and an ambient optical medium formed by air (n_{SE}=1), any angle of incidence of source light larger than a threshold angle of 41.8° on this interface would lead to total reflection. Source light with a smaller angle of incidence is partly reflected to a rather small extent at the outer face of the cover plate, as governed by the well-known Fresnel equations.

Thus, the propagation path of the second source light fraction can be determined in the design process of the PPG device, and the optical blocking arrangement can be positioned suitably inside the casing so as to block the propagation path. In a first alternative embodiment, the optical blocking arrangement is thus arranged to block light incident on the outer face of the cover plate from inside the casing before total reflection at the outer face of the cover plate can occur. That is, it blocks the second source light fraction formed by source light having an angle of incidence larger the threshold angle of total reflection. In a second alternative embodiment, the optical blocking arrangement is arranged to block the second source light fraction formed by a fraction of source light that has been subject to total reflection at the outer face of the cover plate. In both alternative embodiments, an unwanted reduction of the first source light fraction is suitably avoided by proper mutual positioning of the light source, light sensor and optical blocking arrangement, also taking into account the geometrical design of the casing.

The optical blocking arrangement may be implemented in different ways. In one embodiment, the optical blocking arrangement comprises at least one absorption element on an inner face of the cover plate facing the at least one light source and the at least one light sensor. The absorption element is made of a material suitable for absorbing impinging source light and has a dull surface that suitably exhibits no reflection of source light or at least has a low reflection coefficient for the source light. The absorption element may for instance be formed by one or more stripes printed or otherwise applied to the inner face of the cover plate. The absorption element of this variant can be produced for example by a mechanical printing process. In a further variant, the absorption element is applied to the inner face of the cover plate by a gluing process. In a further variant, the absorption element is formed by a dull section of the inner face of the cover plate that faces the at least one light sensor and the at least one light source. Furthermore, if applied to the inner face of the cover plate, the absorption element can prevent a detection of a source light fraction, which would be reflected on the inner face in absence of the absorption element. Absorption elements can be provided in any desired spatial distribution on the inner face of the cover plate.

In other embodiments the optical blocking arrangement forms an integrated part of the cover plate. In some variants, it is formed by one or more absorption stripes or dull material sections immersed in an otherwise transparent cover plate, which is for instance made of a glass material or another suitable material that is transparent at least for the wavelength of the source light.

Regarding geometry of the absorption element, in a further embodiment the absorption element has a lateral extension in a direction pointing from the light source to the light sensor, wherein the lateral extension equals a thickness of the cover plate. This extension of the absorption element shows particularly good results with respect to a blocking of the second source light fraction and avoids an unwanted blocking of the first source light fraction.

In some embodiments, a plurality of light sources is provided to increase the overall intensity of the desired first source light fraction. The light sources are preferably arranged in pairs with a corresponding number of light sensors, such that each light source is assigned to a respective light sensor. A corresponding number of absorption elements may then be used, each having a respective extension in the direction that points from a respective light source to the assigned light sensor in correspondence with the thickness of the cover plate. Thus, the absorption elements are advantageously optimized for the respective pairs formed by a light source and an assigned light sensor.

A full separation of the propagation paths of the first source light fraction and of the second source light fraction is not necessary, even if it is of course advantageous and preferred to provide for a detection of as much of the first source light fraction as possible. In some embodiments, the optical blocking arrangement blocks a portion of the first source light fraction along with the desired blocking of the second source light fraction. However, other embodiments reduce such partial blocking of the first source light fraction by a suitable geometrical design and arrangement of the light source, cover plate and the light sensor. Other embodiments of the PPG device make use of the polarization properties of light and the depolarizing effects of scattering by biological tissue to block the propagation of the second source light fraction. Specifically, scattering of incident polarized light by biological tissue depolarizes the light, and thus the detected backscattered light is substantially unpolarized. On the other hand, polarized source light that is backscattered or reflected by inorganic material used for the cover plate does not lose its high degree of polarization.

In an embodiment of the PPG device therefore, the light source is configured to provide the source light with a first polarization. The optical blocking arrangement comprises a polarization filter in the propagation path of the second source light fraction between the cover plate and the at least one light sensor, i.e., after scattering or reflection of the source light by the cover plate towards the light sensor. The polarization filter is configured to block a propagation of source light of the first polarization, implying that it is at the same time configured to allow a transmission of source light of a polarization other than the first polarization. Source light depolarized by scattering at the external object includes at least a light portion which has a polarization other than the first polarization.

The first polarization is in different variants of this embodiment a linear, circular or ellipsoidal polarization of the source light. The light source is in some variants configured to emit polarized light.

A suitable light source in the PPG device generally is a light emitting diode (LED) or a laser diode (LDs). For embodiments making use of polarization for blocking the second source light fraction, LEDs or LDs emitting polarized light can be used. However, in other embodiments, the light source has a light emitter which emits unpolarized source light. For making use of such unpolarized light emitters in the light source of embodiments using polarized light, the emitted source light is polarized after emission by the light by passing through a polarization filter arranged between the light emitter and the cover plate. In such variants, therefore, two polarization filters of mutually exclusive polarizations are preferably used, a first polarization filter for polarizing the source light, and a second polarization filter for selectively blocking the second source light fraction and allowing a transmission of at least a portion of the first source light fraction carrying the vital-signal information.

The direction polarization of the first and second polarization filter are in some embodiments linear directions of polarization which are mutually orthogonal. The first source light fraction, which has been depolarized by scattering at the external object, can partly pass through the second polarizing filter in front of the light sensor. It is noted that total reflection of the second source light fraction at the outer face of the cover plate may influence the direction of polarization of the second source light fraction. This can be taken care of by using a polarization filter that blocks the largest amount of the second source light fraction.

The use of absorption elements and polarization filters can be combined to further improve the suppression of detection of the second source light fraction. Thus, some embodiments have an optical blocking arrangement that comprises the polarization filter and further comprises at least one absorption element as described before, for instance on the inner face of the cover plate.

A respective polarization filter of the first and second polarization filters is suitably arranged in front of the light source and in front of the light sensor, respectively of the PPG device. This way, the first polarization filter filters all light emitted by the light source and the second polarization filter filters all light propagating towards the light sensor. More specifically, the second polarization filter is arranged, with respect to the propagation path of the second source light fraction, in front of the light sensor. Of course, where more than one light source and/or more than one light sensor is used, additional first polarization filters are employed to provide all source light with the first polarization, and additional second polarization filters are used to fully block the second source light fraction.

In further embodiments, a separation wall is arranged between the light source and the light sensor and blocks a third source light fraction propagating from the light source directly to the light sensor. The at least one separation wall of this embodiment can help to prevent an undesired detection of a further component of source light that has not left the casing of the PPG device and therefore does not contribute to the useful signal to be detected by the light sensor.

In a further embodiment, a protection layer, which is transparent for the source light, is used to embed the at least one light source and/or the at least one light sensor above a base plate on which the light source and light sensor are attached. The protection layer is preferably made of an elastically deformable material, for instance a silicone material. It helps protecting the at least one light source and the at least one PPG sensor against physical impact, occurring for instance when a person wearing the PPG device on its arm like a watch is running or doing other kinds of sports. It also protects from contact with liquids, like sweat or water. Embodiments of the PPG device may therefore also be used under water, keeping in mind that care has to be taken of changed optical properties due to propagation of light in water when designing the positioning of the optical blocking arrangement.

The at least one light sensor is suitably a photodiode sensitive in the spectral range of the light source. However, any other light-sensitive detector device can be used, including a CCD sensor, or a video camera.

It shall be understood that a preferred embodiment of the present invention can also be any combination of the dependent claims or above embodiments with the respective independent claim.

These and other aspects of the invention will be apparent from and elucidated further with reference to the embodiments described hereinafter.

### BRIEF DESCRIPTION OF THE DRAWINGS

In the following drawings:

FIG. 1 shows a schematic illustration of an embodiment of a PPG device;

FIG. 2 shows a schematic illustration of another embodiment of the PPG device, which has an optical blocking arrangement that comprises absorption stripes;

FIG. 3 shows a diagram, which illustrates a dependency between a power of unwanted source light that reaches a light sensor directly after a reflection at the cover plate, for an embodiment of a PPG device;

FIG. 4 shows a schematic illustration of an embodiment of the PPG device, which has an optical blocking arrangement that comprises a plurality of polarizing filters;

FIG. 5 shows an illustration of a first embodiment of the PPG device that is arranged in a watch.

### DETAILED DESCRIPTION OF EMBODIMENTS

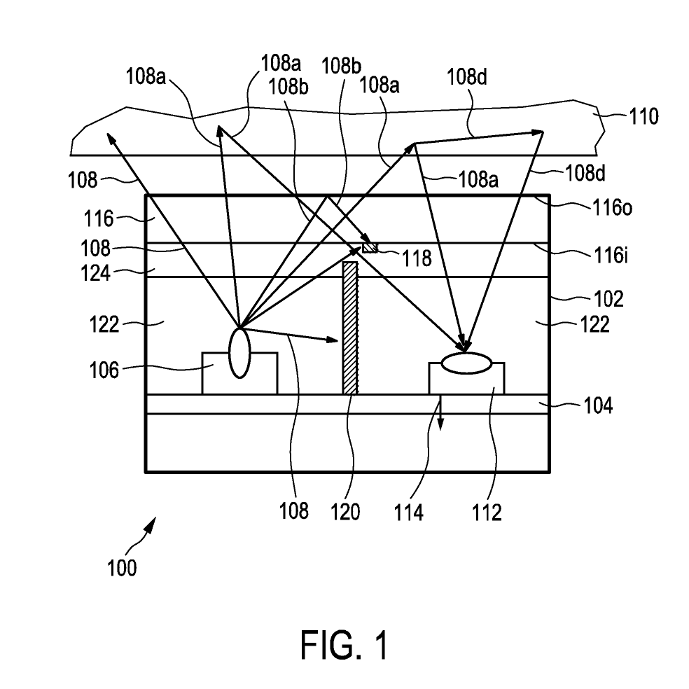

FIG. 1 shows a schematic illustration of an embodiment of a photoplethysmography (PPG) device 100. The drawing is not to scale and the extensions of the individual structural elements are not intended to be indicative of actual extensions and relations between extensions of different structural elements. The drawing is also schematic in that components not required for elucidating features in the context of the present embodiment are omitted. In particular, electrical interconnections are not illustrated.

The PPG device 100 comprises a casing 102. A base plate 104 such as a circuit board with mounted electronic components is arranged in the casing 102. As one of the electronic components, a light source 106 is provided on the base plate 104. are The light source 106 is a light emitting diode (LED) that in operation generates source light generally referenced under the label 108 that is at least in part directed towards an external object 110, which for instance is a finger or arm of a user. A number of arrows labelled 108a, 108b, 108c, and 108d are shown to illustrate different fractions of the source light and will be explained further below. The wavelength of the source light is suitable for entering the external object 110 and being scattered in particular by blood inside blood vessels in the external object 110.

A light sensor 112 in the form of a photodiode is arranged on the base plate 104. It is positioned with a lateral distance from the light source 106. The light sensor 112 is sensitive in the spectral range of the source light 108 and thus allows providing at its output 114 an electronic sensor signal that is indicative of an intensity of a first source light fraction 108a, which has been scattered inside the external object 110 towards the light sensor 112 and then detected by the light sensor 112. The first fraction of scattered source light is redirected towards the light sensor 112 by source light 108a and 108d subject to single (108a) or multiple (108d) scattering events inside the external object 110, as indicated by arrows 108a and 108d in a very simplified manner.

The casing 102 housing the light source 106 and the light sensor 112 has a cover plate 116, which is transparent for the source light 108 and which has an inner face 116i facing the light source 106 and the light sensor 112, and an outer face 116o facing the external object 110. The cover plate 116 gives rise to scattering of source light, in particular in the form of reflection of source light. A small fraction of the source light is reflected at the inner face 116i. A non-negligible fraction of the source light, which is herein referred to as the second source light fraction 108b, is reflected at the interface between the outer face 116o of the cover plate 116 and the ambient air outside the casing 102, and is redirected towards the light sensor 112 without having left the casing 102.

An optical blocking arrangement 118 is arranged in the casing 102 between at the inner face 116i of the cover plate 116. It blocks the propagation path of the second source light fraction 118b on its way from the light source 106 light sensor 112 after reflection at the outer face 116o of the cover plate 116. The optical blocking arrangement 118 of the present embodiment is an absorption stripe applied to the inner face 116i of the cover plate 116. At least a portion of source light that is reflected at the inner face 116i of the cover plate 116 can be blocked by the optical blocking arrangement 120 of this embodiment by proper design of the width of the optical blocking arrangement. To this end, in addition to providing a material with particularly high absorption of the source light, the absorption stripe is made of a material with particularly low reflectance.

A separation wall 120 serves to block a fraction 108c of the source light, which herein is also referred to as the third source light fraction and would in absence of the separation wall directly propagate from the light source 106 to the light sensor 112. The use of a separation wall can be avoided by providing the light source with a sufficiently small beam aperture of the emitted source light, which can for example be achieved with a lens as an optical collimation element. The lens can be incorporated as an integral part into the light source.

A protection layer 122 made of a silicone, which is transparent for the source light 108, covers the light source and the light detector.

Thus, while the casing 102, supported by the silicone layer 122, protects the light source 106 and the light sensor 112 against physical damage, a useful component of the light sensor signal corresponding to the first source light fraction 108a, 108d, which carries the vital-sign information, is provided with particularly small perturbation, despite the use of the cover plate. This eliminates or at least reduces an intensity of the unwanted second source light fraction 108b that does not reach the external object but—in absence of the optical blocking arrangement 118—would have reached the light sensor 112 without leaving the casing 102, in particular after reflection at the outer face 116o of the cover plate 116 of the casing 102. This elimination or at least reduction of the intensity of this second source light fraction 108b in the detected signal increases the relative amount of the useful first source light fraction 108a, 108d, which has been scattered by the external object 110 and thus contains desired vital-sign information when it is detected by the sensor 112. This allows extracting and evaluating the vital-sign information with high accuracy.

The most suitable position and shape of the optical blocking arrangement 118 can determined by simulation of the optical pathways during the design process, in consideration of the geometry and arrangement of the light source 106 and the light sensor 112 and the light propagation inside the casing 102, including light propagation inside the cover plate undergoing multiple reflections at transitions from the cover plate to an adjacent air space 124 inside the casing 102 and to ambient air outside the casing 102.

A PPG processing unit (not shown) may be provided either inside or outside the casing and receive the detection signal from the output 114 of the light sensor 112 for processing and determining the vital-sign information.

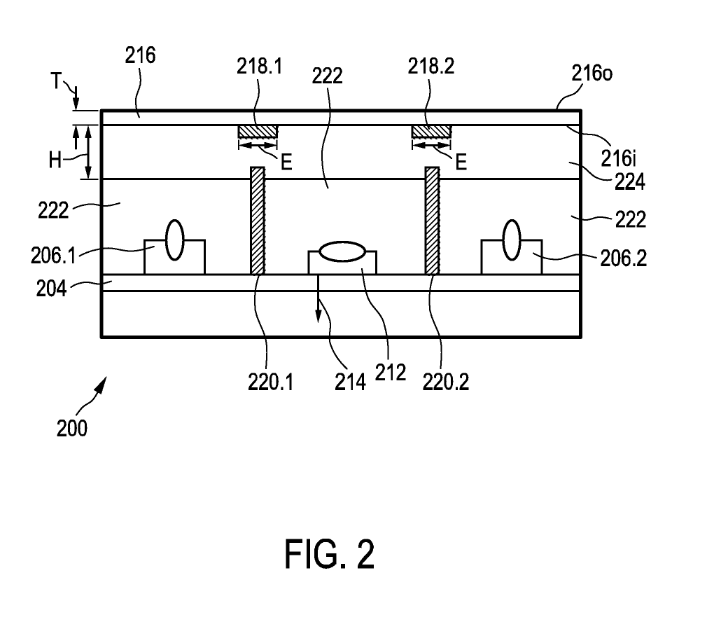

FIG. 2 shows a schematic illustration of a second embodiment of a PPG device 200.

The following description focuses on differences between the embodiments of FIGS. 1 and 2. Reference labels differing only in the first digit are used to indicate components of the PPG device 200 which are also comprised by the embodiment of the PPG device 100 of FIG. 1. Reference is thus additionally made to the description of FIG. 1 for such features, unless differences are explained in the following.

The PPG device 200 of FIG. 2 has two light sources 206.1 and 206.2, which in the present case are identical in their physical characteristics, in particular in their emission wavelength. This increases the scattered source light intensity and thus improves the accuracy of the vital-sign information to be determined. However, it is not a requirement that only light sources with identical physical characteristics are used. For instance, different wavelengths of source light may be used to obtain different types of vital-sign information, such as pulse frequency and blood oxygen saturation.

The first and second light sources 206.1 and 206.2 are arranged to the left and to the right of the light sensor 212 on the base plate 204. Separation walls 220.1 and 220.2 are provided between the first and second light sources 206.1 and 206.2, respectively, and the light sensor 212.

The optical blocking arrangement comprises absorption elements 218.1, 218.2 in the form of absorption stripes. The absorption elements 218.1 and 218.2 are printed on the inner face 216i of the cover plate 216 and of a black color and a dull surface.

The first absorption element 218.1 is printed on the inner face 216i in a lateral position that is substantially in the middle between the first light source 206.1 and the light sensor 212. The second absorption element 218.2 is printed on the inner face 216i in a lateral position that is substantially in the middle between the second light source 206.2 and the light sensor 212. The positions are selected such that the absorption elements 218.1 and 218.2 block a propagation of the described second source light fraction from the outer face 216o of the cover plate 160 to the light sensor 212. At the same time the absorption elements 218.1 and 218.2 avoid a reflection of source light at the inner face 216i of the cover plate 216.

Both absorption stripes have an extension E in a direction parallel to the inner surface of the cover plate and in the paper plane of FIG. 2 which substantially equals a thickness T of the cover plate 216. The thickness T of the cover plate 160 is typically between 0.3 mm and 1.5 mm. The air gap 224 between the silicone layer 222 and the cover plate 116 has a height H which is in some embodiments between 0.02 mm and 0.15 mm.

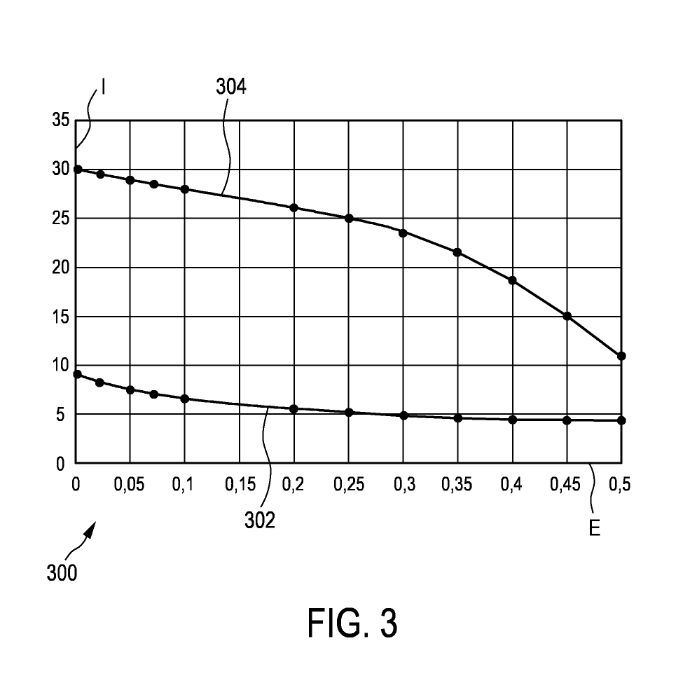

The extension E of the absorption elements 218.1 and 218.2 is suitably determined on the basis of simulation results discussed in the context of FIG. 3 below.

FIG. 3 shows a diagram 300, which illustrates a dependency between a relative amount I of the unwanted second source light fraction that reaches the light sensor 212 and the stripe width E for an embodiment of a PPG device substantially corresponding in construction to that shown in FIG. 2.

The relative intensity amount of the unwanted second source light fraction reaching the light sensor 130 is plotted on the ordinate in units of % of the total emitted source light intensity. The extension E of the absorption elements 218.1 and 218.2 is plotted on the abscissa in linear units of millimeter. The thickness T of the cover plate 216 underlying the simulation is 0.5 mm and the height H of the air gap 224 is 0.1 mm. A lateral distance between the light source and the light sensor is about 1.5 mm. Two different scenarios are represented by two simulation curves 302 and 304. The scenario underlying the curve 302 is a reference scenario, in which there is no space (and thus no air) between the outer face of the cover plate and skin of a user (forming the external object in the language of the claim). The scenario underlying the curve 304 has an air gap between the outer face of the cover plate and skin of the user. The simulations were calculated assuming a user having white skin.

The extension of the absorption elements in the direction perpendicular to the paper plane is assumed to be identical and sufficient for blocking any light that reaches a lateral position along the extension E in the paper plane.

As determined by the simulation and visible in FIG. 3 from the curve 304, the relative amount of unwanted light (second source light fraction) detected by the light sensor 130 can be reduced by a factor of at least about 3 by using the absorption stripes 212, 214 with a stripe width E of 0.5 mm, while the reference scenario exhibits a reduction by a factor of about 2 only.

It is noted that simulations done with dark skin using the same scenarios show that the received second source light fraction, which may also be called “shortcut light”, is in fact equal to the useful signal formed by the first source light fraction, when no absorption stripes 212, 214 are used. In other words, in this case the curve 304 would start at I=50% for a stripe width E=0 mm. Still, in that case, for a stripe width of 0.5 mm, the relative intensity of the “shortcut light” can be reduced to 10%. As a rule of thumb therefore, the strip width is preferably chosen equal to the thickness of the cover plate. In general, for a given sensor, proportionality is observed between the stripe width suitable for achieving a relative intensity I of the “shortcut light” of 10% and the thickness T of the cover plate. The thicker the cover plate, typically a glass plate, is, the wider the stripe width E has to be.

The suitable stripe width E for achieving I=10% also depends on a lateral distances between the light source and the light sensor. As a rule of thumb, in comparison with the simulation data given above, an increase of the lateral distance by a factor F results in a suitable stripe width E that is equally increased by the factor F in comparison with the values presented for the simulations above. As an example, if the lateral distance is increased by a factor F=3 in comparison to the example of FIG. 3, then the suitable is preferably at E=3T.

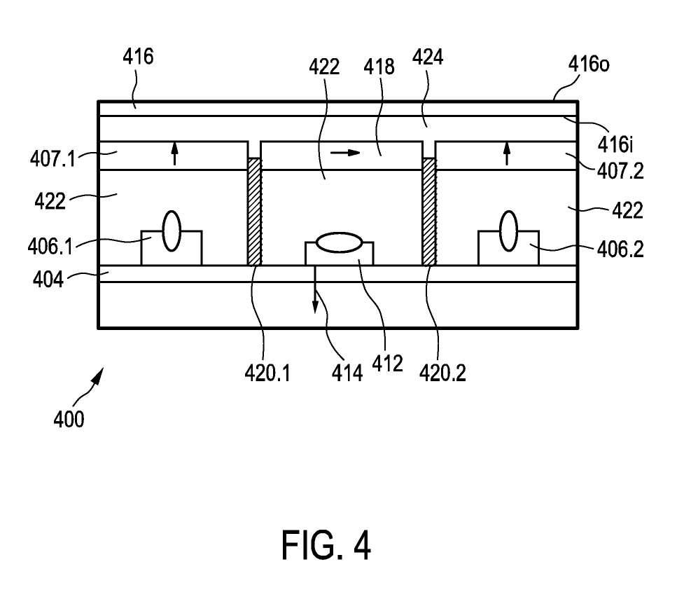

FIG. 4 shows a schematic illustration of a further embodiment of a PPG device 400. The following description focuses on differences between present embodiment and the embodiments of FIGS. 1 and 2. Reference labels differing only in the first digit from those used in FIGS. 1 and 2 are used to indicate components of the PPG device 400 which are also comprised by the embodiment of the PPG device 100 of FIG. 1 or the PPG device 200 of FIG. 2. Reference is thus additionally made to the description of FIG. 1 and FIG. 2 for such features, unless differences are explained in the following.

The PPG device 400 of FIG. 4 has two light sources 406.1 and 406.2, which in the present case are identical in their physical characteristics, in particular in their emission wavelength and in the fact that they provide unpolarized light. The first and second light sources 406.1 and 406.2 are arranged to the left and to the right of the light sensor 412 on the base plate 404. Separation walls 420.1 and 420.2 are provided between the first and second light sources 406.1 and 406.2, respectively, and the light sensor 412.

Two polarization filters 407.1 and 407.2 are provided on top of the silicone layers 422 and the light sources 406.1 and 406.2, respectively. The have also been referred to as first polarization filters and serve to provide polarized source light. In the present example, both polarization filters 407.1 and 407.2 allow the transmission of linearly polarized light with a first direction that is only schematically indicated in FIG. 4. As is well known, the polarization vector of a light wave is in a plane that is perpendicular to the direction of propagation of the light wave. The actual direction of polarization of the source light provided behind the polarization filters 407.1 and 407.2 therefore points in a direction perpendicular to the paper plane of FIG. 4. The optical blocking arrangement 418 comprises a further polarization filter, which has been referred to as the second polarization filter above. The polarization filter 418 is arranged on top of the light sensor 412 and the silicone layer 422. It allows transmission only of linearly polarized light that has a polarization vector in the paper plane of FIG. 4 and parallel to the cover plate 416. In particular, the allowed polarization direction of the second polarization filter 407.2 is orthogonal to the allowed polarization direction of the first polarization filter 407.1.

The PPG device 400 makes use of the depolarizing effects of scattering by biological tissue to block the propagation of the second source light fraction by the second polarization filter 418. Specifically, since scattering of incident polarized light by biological tissue depolarizes the light, and thus the detected backscattered light is substantially unpolarized, polarized source light that is backscattered or reflected by inorganic material used for the cover plate can be filtered out by the second polarization filter 418 because it has not lost its high degree of polarization achieved by the first polarization filters 407.1 and 407.2. By providing the optical blocking arrangement in the form of the polarization filter 418 in the propagation path of the second source light fraction between the cover plate 416 and the light sensor 412, i.e., after scattering or reflection of the source light by the cover plate 416 towards the light sensor, a blocking of the propagation of source light of the polarization induced by polarization filters 407.1 and 407.2 is achieved. On the other hand, source light depolarized by scattering at the external object includes at least a light portion of roughly 50% of its intensity which does not have the polarization induced by polarization filters 407.1 and 407.2. This portion also contains the desired signal and thus the vital-signal information.

In alternative embodiments not shown, the polarizing filters allow a passing of source light of respective first and second mutually exclusive circular or ellipsoidal polarizations.

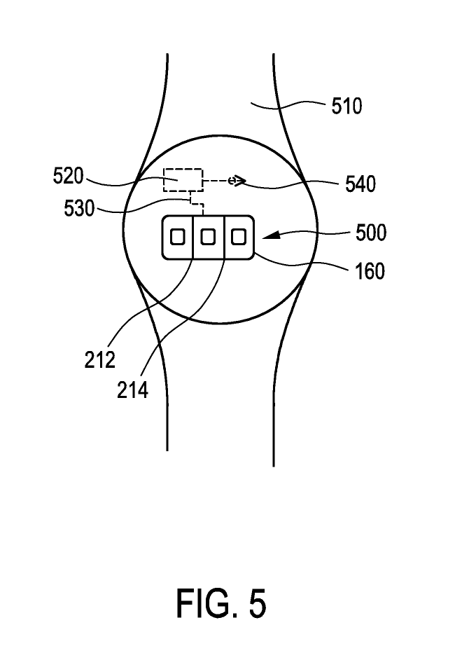

FIG. 5 shows an illustration of a first embodiment of the PPG device 500 that is arranged in a watch 510. The only difference between the PPG device 500 and the PPG device 200 with the absorption elements 218.1, 218.2, as shown in FIG. 2, is that the PPG device 500 is arranged in the watch 510 and connected with a processing unit 520.

The processing unit 520 is configured and arranged to receive the sensor signal 530 and to process the sensor signal 530 in order to provide a PPG information signal 540 indicative of PPG characteristics, which can be displayed by the watch 510 upon a respective activation of the PPG device 500 by a user of the watch 510.

The cover plate 160 of the PPG device 500 protects the PPG device 500 against physical contact with the user as well as against moisture of the arm or wrist of the user.

In summary, the invention relates to a photoplethysmography device that comprises a light source configured to direct source light towards an external object; a light sensor arranged and configured to provide a sensor signal indicative of an intensity of a first source light fraction, which has been scattered by the external object; a casing for housing the light source and the light sensor, and having a cover plate transparent for the source light and an outer face to be facing the external object; and an optical blocking arrangement in the casing between the at least one light source and the outer face of the cover plate and configured to block a second source light fraction on its propagation path extending from the light source to the outer face of the cover plate and from the outer face of the cover plate to the light sensor without leaving the casing.

While the present invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims.

In particular the invention is not restricted to the use of a whole medical apparatus containing a PPG processing unit or to monochromatic light sources. The invention is furthermore not restricted to medical applications.

In the claims, the word “comprising” does not exclude other elements or steps, and the indefinite article “a” or “an” does not exclude a plurality.

A single step or other units may fulfill the functions of several items recited in the claims. The mere fact that certain measures are recited in mutually different dependent claims does not indicate that a combination of these measures cannot be used to advantage.

Any reference signs in the claims should not be construed as limiting the scope.

1. A photoplethysmography device, hereinafter PPG device, comprising:
at least one light source configured to generate a beam of source light to be directed towards an external object;
at least one light sensor which is arranged and configured to provide a sensor signal indicative of an intensity of a first source light fraction, which has been scattered by the external object and detected by the light sensor;
a casing housing the at least one light source and the at least one light sensor, the casing having a cover plate, which is transparent for the source light and which has an outer face to be facing the external object; and
an optical blocking arrangement, which is arranged in the casing between the at least one light source and the outer face of the cover plate and which is configured to block a second source light fraction on its propagation path extending from the light source to the outer face of the cover plate and from the outer face of the cover plate to the light sensor without leaving the casing.

2. The PPG device according to claim 1, wherein the optical blocking arrangement is arranged to block the propagation path of the second source light fraction propagating from the outer face of the cover plate to the light sensor.

3. The PPG device according to claim 1, wherein the optical blocking arrangement comprises at least one absorption element on an inner face of the cover plate facing the at least one light source and the at least one light sensor.

4. The PPG device according to claim 1, wherein the optical blocking arrangement forms an integrated part of the cover plate.

5. The PPG device according to claim 3, wherein the at least one absorption element has a lateral extension in a direction pointing from the light source to the light sensor, and wherein the lateral extension equals a thickness of the cover plate.

6. The PPG device according to claim 1, wherein the light source is configured to provide the source light with a first polarization, and wherein the optical blocking arrangement comprises a polarization filter in the propagation path of the second source light fraction between the cover plate and the at least one light sensor, the polarization filter being configured to block a propagation of light of the first polarization.

7. The PPG device according to claim 6, wherein the light source comprises a light emitter configured to emit unpolarized light and a first polarization filter that is configured to provide the source light with the first polarization, and wherein the polarization filter of the optical blocking arrangement forms a second polarization filter exclusively allowing a transmission of source light having a second polarization, the first and second polarizations being mutually orthogonal.

8. The PPG device according to claim 7, wherein the second polarization filter is arranged, with respect to the propagation path of the second source light fraction, in front of the light sensor.

9. The PPG device according to claim 8, wherein a separation wall is arranged between the light source and the light sensor and blocks a third source light fraction propagating from the light source directly to the light sensor.

10. The PPG device according to claim 9, further comprising a silicone layer, which is transparent for the source light and arranged on a base plate, and which surrounds those sides of the at least one light source and of the at least one light sensor that are not attached to the base plate.
